(19) United States
(12) Patent Application Publication
(10) Pub. No.: US 2018/0108437 A1
(43) Pub. Date: Apr. 19, 2018

(54) METHOD FOR CREATING A PERSONALIZED MEMORY COMPILATION FOR COLLABORATIVE THERAPEUTIC REMINISCENCE

(71) Applicant: Applaud, LLC, Cincinnati, OH (US)

(72) Inventors: Robert R. Reinders, Cincinnati, OH (US); Christopher Romano, Cincinnati, OH (US)

(21) Appl. No.: 15/843,972

(22) Filed: Dec. 15, 2017

Related U.S. Application Data
(63) Continuation of application No. 14/191,430, filed on Feb. 27, 2014, now abandoned.
(60) Provisional application No. 61/770,271, filed on Feb. 27, 2013.

(51) Int. Cl. G06Q 50/00

(52) U.S. Cl. CPC G16H 20/70; G06Q 50/01

(57) ABSTRACT

The present invention may provide a method and apparatus for generating a Personalized Memory Compilation, soliciting memories from members of a first group, collecting content from one or more data for the memories into a content collection, identifying preferences of the members of the first group, analyzing portions of the content collection to identify collaborative memories presenting the collaborative memories to a user for indication of social support, developing latent memories from collaboration between group members, reconsolidating memories from the collaborative memories and the developed latent memories, and compiling a collaborative output from the content. The method may include steps of increasing or decreasing social support or altering perceived social support.

### CROSS-REFERENCE TO RELATED APPLICATION

The application is a continuation of U.S. patent application Ser. No. 14/191,430 filed Feb. 27, 2014, which is based on and claims priority from U.S. Provisional Patent Application No. 61/770,271, filed on Feb. 27, 2013, both of which are hereby incorporated by reference in their entirety for all purposes.

### BACKGROUND OF THE INVENTION

The present invention relates generally to creation and/or publishing of personalized memory compilations for members of a group and, more particularly, to a method for gathering, enriching, preserving, and sharing memories for members of a group using the combination of 1) collaboration through nominal group recall and 2) recording of personal preferences to produce a digital or printed asset that contains a blend of selected profiles, images, stories, personalized notes, and other relevant content from more than one group member and constitutes rendering of an important experience of the group.

Life's most meaningful events are often shared experiences with a group of family or friends. Participants have a strong desire to preserve and share the memories of these milestone occasions, as evidenced by the more than 50 billion digital photos taken last year; an increase of about 10 billion over just a few years ago.

The success of online social networks demonstrates that people want to preserve and share their stories in their own special way. They often care what others think, demonstrate a strong desire for personalization and employ technology with ease.

Many consumers demand personalized interaction. With the advent of high speed, high quality, digital print engines and software that can integrate fixed and variable content, variable data communication (VDC)—also called cross media communication—represents a major shift in technology and strategy to provide highly relevant, one-to-one communication with customers. Cross media communication represents advanced techniques and supporting technology that helps producers and marketers keep pace with customers' expectations regarding media. Many consumers demand personalized interaction. VDC enhances the value of communication by making the level and depth of conversation personal and powerful. Although measures of return on investment vary according to the selected media, response rates for VDC are often much higher than static media.

This major shift in technology has served to stimulate a high-growth market for custom photo books. On-demand publishers allow authors or photographers to print factory-bound, hard and soft cover books from a digital source for a low price. Consumers upload their photos and descriptions, and then print as many or as few books as they want. From a time and cost-saving perspective, these services have become useful for people who used to create their own photo albums and scrap books. Now they can save money by not printing individual photographs or purchasing albums and scrapbooking materials. Still, with photo book providers' current offerings, creating a unique photo book for more than one member of a group requires an extensive commitment of time, energy, and money.

The present invention addresses the challenge of collecting images and stories from a group of people, then assembling them as a personalized memory compilation in a way that preserves memories by: gathering and blending stories; images and other associated content from members of a group; creating a series of custom memory compilations where each one may be distinct and personalized to the group member; and replicating the process for a number of groups simultaneously.

### SUMMARY OF THE INVENTION

In one aspect of the present invention, a method to build a memory compilation for members of a group comprises; soliciting memories from members of a first group, collecting content from one or more data for the memories into a content collection, identifying preferences of the members of the first group, analyzing portions of the content collection to identify collaborative memories presenting the collaborative memories to a user for indication of social support, developing latent memories from collaboration between group members, reconsolidating memories from the collaborative memories and the developed latent memories, and compiling a collaborative output from the content collection.

These and other aspects, objects, features, and advantages of the present invention, are specifically set forth in, or will become apparent from, the following detailed description of an exemplary embodiment of the invention when read in conjunction with the accompanying drawings.

### BRIEF DESCRIPTION OF THE DRAWINGS

FIG. 1 is a plan diagram of group members sharing information separately, according to an embodiment of the present invention;

FIG. 2 is a plan diagram of group members' memories selectively including information among selected group members, according to an embodiment of the present invention;

FIG. 3 is a plan diagram of selected group members collaboratively sharing information, according to an embodiment of the present invention;

FIG. 4 is a plan diagram of an isolated portion of FIG. 3, according to an embodiment of the present invention;

FIG. 5 is a plan diagram of group members from FIG. 3, with retained enhanced memories, according to an embodiment of the present invention; and

FIG. 6 is a flowchart of a method, according to another embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Connectedness describes the degree to which a person or group is socially close, interrelated, or shares resources with other persons or groups. This concept concerns the nature and quality of connections both within and between multiple levels of the social ecology, including: i) connectedness between individuals; ii) connectedness of individuals and their families and friends to community organizations; and iii) connectedness among community organizations and social institutions.

Feeling connected is a fundamental human desire. Humans are driven by primal motivations and are wired to get connected and stay connected with other people, including friends and family. Some of this social instinct may be traced back hundreds of millions of years, to the first mammals. Other parts of the social mind are more modern. Recent advances in the fields of affective neuroscience and social cognitive neuroscience help to explain, and harness, these universal, cerebral mechanisms to better lives. Such advances may significantly help people who are struggling with illness or injury, as well as those who simply desire to connect with others on a deeper and more meaningful level.

Received or perceived social support may decrease the threat-level appraisal of experienced stress and increase a person's ability to cope with a stressful event or situation. Close and supportive interpersonal relationships also appear to confer general psychological benefits independent of stress that increase physiologic functioning, such as cardiovascular, endocrine, and immune systems. Overall health may improve and resistance to stress and disease may be enhanced. Close and supportive interpersonal relationships may also help to discourage maladaptive coping activities such as suicidal behavior or substance abuse, and by virtue of normative social influences, encourage adaptive coping behaviors such as seeking therapy or other professional help.

In one embodiment of the present invention, a method and system may facilitate therapeutic expression of memories—enabling groups of people to gather, preserve, enrich and share memories—to help people to reach a deep level of connection with others. Memories are important—a link to the past and a source of knowledge that shapes life choices. Memories may be influenced dramatically by language and images, and may be closely tied to emotions and mental health. Recalling and sharing may help to affirm one's identity and bring people closer together.

Reminiscing may be a purposeful act of recalling memories. Memories may be expressed explicitly, as in conscious recollection, or implicitly, as in performing a task without conscious recollection but with information acquired during previous study or action. When groups of people reminisce together, latent memories may emerge. Latent memories are memories that are not presently obvious or active, but are capable of emerging as a result of information learned or recalled from sharing. Latent memories may affect the recall process from the capacity to alter how explicit and implicit memories are perceived and later recalled.

Reminiscence may be used therapeutically to treat physical or emotional illness or injury. By soliciting explicit and implicit memories, therapeutic reminiscence may provide a structured and enjoyable activity; promote intrapersonal and interpersonal functioning; stimulate cognition, memories and emotions; and improve indicators of well being. For example, therapeutic reminiscence may be beneficial for people undergoing physical, cardiac or cognitive rehabilitation. Reminiscence may also be used to improve well being while minimizing illness or injury. For example, therapeutic reminiscence may be used to strengthen military families for the physical, psychological and emotional rigors of deployment far from home.

Collaborative Therapeutic Reminiscence (CTR) is a structured method for collaboratively conducting therapeutic reminiscence with a group of people, and also curating the creation of a Personalized Memory Compilation (PMC). CTR may involve coordinating a series of processes such as nominal group recall, selective inclusion, latent memory elicitation, and memory reconsolidation with a group of people who have a shared experience. CTR may be performed face-to-face or electronically using online software or a software application installed on a computer or mobile device (sometimes called an “app”), or any other suitable method.

PMC may be a personalized, therapeutic tool that helps with the process of memory reconsolidation. PMC may enhance outcomes from therapeutic reminiscence by facilitating recall of past experiences and contributing to latent memory elicitation and memory reconsolidation; removing obstacles to the primary therapeutic objectives; and enhancing support and connectedness between participants. Created iteratively during CTR, PMC may contain a variety of personalized and user-centric content obtained from group members during recall sessions. PMC may be generated by using software that gathers, selects, assembles, enriches and preserves contributions from group members; whether the group is an established group or an ad-hoc group. PMC may constitute an artifact of CTR that facilitates recall for participants, and facilitates sharing the result of the activity with people who are not part of the intended group.

In the field of preventive health, a number of evidence-based protective factors have been identified including optimism, problem-solving, self-efficacy, impulse control, spirituality, resilience, and close relationships (or social support). In general, resilient or hardy individuals are thought to use active coping mechanisms when dealing with stressful life situations. Receiving community and familial support during times of significant stress may promote mental or emotional resilience (the capacity to adapt or change successfully in the face of adversity) and self-efficacy (belief in one's ability to handle difficult situations or perform well), which may strengthen coping abilities and mitigate the negative psychological consequences of stressors.

Evidence is growing in the medical literature about social support and the neurobiological pathways through which social support acts to foster resilience and reduce the risk for developing physical and emotional illness. Positive social support may enhance resilience to stress, help protect against developing trauma-related psychopathology, decrease the functional consequences of trauma-induced disorders, such as post-traumatic stress disorder (PTSD), and reduce medical morbidity and mortality. The evidence is strong that poor social support has harmful consequences and having access to rich and functional social networks has a protective effect on maintaining physical and psychological health.

Social isolation and low levels of social support have been shown to be associated with increased morbidity and mortality in a host of medical illnesses. In human studies, low social support has been associated with physiological and neuroendocrine measures of heightened stress reactivity, including elevated heart rate, increased blood pressure, and exaggerated cardiovascular and neuroendocrine responses. Epidemiological studies have reported that poor social support is associated with the onset and relapse of depression, negative treatment response to dysthymia, seasonality of mood disorder, and the presence of depression in several medical illnesses, such as multiple sclerosis, cancer, and rheumatoid arthritis.

In contrast to low social support, high levels of social support appear to buffer or protect against the full impact of mental and physical illness. The relationship between positive social support and superior mental and physical health has been observed in diverse populations, including college students, unemployed workers, new mothers, widows, and parents of children with serious medical illnesses. Strong social support has been shown to be an important factor in decreasing functional impairment in people with depression and in increasing the likelihood of recovery. Moreover, studies with Vietnam War veterans found the risk of developing PTSD upon exposure to combat trauma is inversely correlated with social support.

Reminiscing is the purposeful act of recalling memories. Memories may be expressed explicitly, as with conscious recollection, or implicitly, as with performing a task without conscious recollection but with information acquired during previous study or action. Compared to individual recall, collaboration increases the confidence, completeness and accuracy of recall. However, collaborating groups do not recall as much as would be gained from combining the non-redundant product of individual participants. The information produced by a group remembering together—although more than any one of the members could have produced alone—is typically less than what is produced by the nominal group (such as, the pooled sum of information produced by the same individuals alone). Moreover, group recall can influence subsequent memory recall of individual group members, affecting accuracy and completeness. Additionally, relationships among group members affect how much and how well each individual remembers.

Nominal group recall is a technique that accounts for how remembering with others is different from remembering alone. Nominal group recall enables groups to produce more information and more accurate information, but takes into account that real groups recall less than nominal groups. Nominal group technique overcomes several limitations and produces a highly reliable, pooled sum of information produced by individual participants.

Selective inclusion refers to the process of choosing the contents of the PMC, either by active selection, or conversely, by passive exclusion. Participants may undergo an interview-style process of identifying their preferences, which are later used to identify portions of content, to be included in the participant's PMC. Preferences affect who is invited to participate, what type of content is requested from participants, the type and quantity of content provided by the primary participant, and the style of the final output of PMC (such as, digital download, personalized website, or printed book). It may be helpful to think of this process as “choosing your friends” from among the group of participants, since who is chosen has a substantial affect on the final makeup of the PMC.

In humans, memory retrieval is not a passive phenomenon, but a process that either reinforces or alters stored information. Long-term memories may be activated, modified, and reconsolidated. Cued recall of a memory is an event that may initiate a memory reconsolidation cascade. Reconsolidation acts to stabilize the expression of the original memory and “update” the original memory with new information to reflect a current state of knowledge.

In complex adaptive systems, networks of interacting individuals create an operating history that weaves together a story. Once that narrative coalesces, knowledge of the narrative by the components of the system can influence individual behavior. Thus, the recall of group memories by group members may be viewed as an evolving dynamical system with group properties that emerge from the interactions among the participants, and between the participants and the group.

Complex systems theories provide a method to perform a mathematical analysis of the non-linear, emergent properties of memories. The fractal function, Zₙ₊₁=Zₙ²+C, represents the group recall of a shared experience among three participants, E, F and G. C represents a catalyst that precipitates the interaction and consequent memory recall. The first iteration (Z₁=E) comes from the first participant E's contribution to the group recall, which consists entirely of explicit memory, those memories recalled directly by the participant.

For a second iteration of the equation (Z₂=Z₁*F+C), the inputs are 1) the second participant F's contribution, also consisting of explicit memory; and 2) the output from the previous function (Z₁). The output this time (Z₂) is a compound of the explicit memory contributed by each individual, including that portion of each participant's explicit memory that intersects with others in the group (those group memories they share).

For the third iteration, and every one thereafter, the participant (in this case, G) may again contribute explicit memory, and the output (Z₃=Z₂*G+C) is the iterated aggregation of all previously contributed explicit memory. The fractal function may continue to iterate for as many participants as required and allow for an unlimited number of participants/iterations. The ultimate output may be a combination of all contributed explicit memories plus an emergent product called latent memories.

Latent memories are not explicit memories, but an emergent function of group recall. Latent memories may arise after participants share their explicit memories. As participants find new information about a shared experience and others provide information, about which individual participants may not recall or about which the individual participants were not previously aware. The significance of latent memory to the task of preserving memories comes from the capacity to alter how explicit memory is both perceived, and later, recalled. A latent memory can come about whether a group member becomes aware of memory-related information by virtue of another group member reminding them (such as, it was something they already knew, but had forgotten) or by virtue of another group member telling them about it for the first time. So regardless of whether it was a new or a “refreshed” latent memory, the emergence portends a change to the group member's explicit memory; demonstrating the non-linear, evolving, emerging nature of group recall (as a complex system).

To preserve a more complete and precise recollection of a shared event, the emergence of latent memory must be carefully managed. If explicit memories are combined among participants too soon in the preservation process, then the development of latent memory may alter recollection by participants. Furthermore, because group recall has non-linear properties, the emergence of latent memory has the potential to change more than one group member's recollection; an outcome potentially deleterious to precision and accuracy.

As group recall takes place, a collaborative memory Zₙ₊₁, is constructed iteratively as each participant contributes explicit shared memory and a latent shared memory may emerge. Using an iterative formula, one can demonstrate the synergistic nature of group recall by comparing the collaborative output to the collective sum of the participants' contributions. Mathematically, as long as catalyst C is positive, a²+2a bi+b²+C will be greater than a². The corollary is, if catalyst C is sufficiently negative—in other words, there is sufficient noise or interference in the process—then a²+2a bi+b²+C could be less than a². In other words, if the process that catalyzes the iterative memory recall is not performed properly (a negative C value), the output could be deteriorated. Conversely, if the process is performed properly (a positive C value), it may produce synergy and symbiosis (such as, an enriched memory.

CTR with a PMC may be valuable to the participants for several reasons. Primarily, it is an evidence-based, purposeful activity that can reduce boredom, increase social engagement, and stimulate cognition and communication. The PMC documents the activity and becomes a “living record” of the participant's recollection. At any point in the activity, progress can be reviewed by the participants or shared with others in a variety of media (e.g., digitally downloaded or printed in color book form) in whole or in parts. Not only is the collection of memories interesting and relevant, but software may perform statistical analysis of activity such as: i) frequency and duration of participation; ii) expansion or contraction of locus of memories over time; iii) occurrence and frequency of repetition or contradiction; and iv) increased/decreased communication. This data may provide family members insight into their loved one's condition they can use to make modifications to their environment or activity.

Reminiscence therapy is a well-established sensory memory-based approach to psychotherapy that is often used to treat people suffering from brain and nervous system damage. Reminiscence therapy may also be used to enhance support and connectedness in post-institution physical rehabilitation. In such cases, the primary goals of reminiscence therapy are to facilitate recall of past experiences to promote intrapersonal and interpersonal functioning, which has been shown to improve well being. In these cases, reminiscence may serve intrapersonal functions, such as enhancing self-understanding and a sense of personal continuity, identifying a sense of meaning to one's life, and facilitating resolution of a life state. Reminiscence also may serve interpersonal functions such as leaving a legacy.

While most branches of health care focus on prevention and treatment of disease, rehabilitation principally focuses on the enhancement of human functioning and quality of life. In a broad sense, rehabilitation attempts to tackle the complex relationship between disease and the ability to function: eradicating disease does not necessarily eliminate disability; likewise, disability can be minimized even in the face of permanent injury or chronic disease.

CTR with a PMC may be used as a method to promote and enhance measures of physical and emotional well being in several types of rehabilitation activities: i) cognitive rehabilitation to help brain-injured or cognitively impaired persons compensate for cognitive deficits; for example in cases of early stage dementia, Traumatic Brain Injury (TBI) or stroke; ii) cardiac rehabilitation to optimize physical function in people with cardiac disease or recent cardiac surgery; iii) physical rehabilitation to treat or prevent disability that may result from diseases, disorders, conditions, or injuries.

CTR with a PMC may be used as a method to promote and enhance measures of physical and emotional well-being in cognitive rehabilitation; for example with people suffering early-stage dementia. Dementia is the loss of brain function that occurs with certain diseases. It is a large and rapidly growing problem. Characterized by impairment in multiple cognitive domains, dementia can affect language, problem solving, judgment, abstraction, movement, and memory. Most types are degenerative and nonreversible. Worldwide, more than 24.3 million people are estimated to have dementia, and 4.6 million new cases are diagnosed each year. The number of people affected is expected to double every 20 years to 81.1 million by 2040. Although Alzheimer's is prevalent, dementia can be brought on by other medical conditions as well as brain injury resulting from physical trauma.

During later stages, people with dementia may become increasingly frail due to the progression of the illness. Some gradually lose their ability to walk and become confined to a bed or chair. Many lose their ability to perform everyday tasks unaided and eventually become dependent on others for all of their care. Memory loss is likely to be severe in the later stages of dementia. People suffering dementia may be unable to recognize those close to them or even their own reflection. They may no longer be able to find their way around familiar surroundings or identify everyday objects. Severe dementia frequently causes such complications as immobility, swallowing disorders, and malnutrition. These complications can significantly increase the risk of developing pneumonia, which has been found in several studies to be the most commonly identified cause of death among elderly people with dementia.

Personally-adapted external aids like a PMC are considered more effective in dementia care because they better meet the patients' capabilities and needs and increase their motivation. In controlled trials, reminiscence involving life review or personalized specific memory triggers in a life-story book showed some psycho-social benefits for people with dementia, whereas no benefits were seen with general reminiscence materials. Additionally, there is strong evidence that a multimedia biography may lead to stimulation and enhancement of memories, enabling participants to better recollect places and names, and that viewings increased familiarity with individuals who appear regularly in a biography.

CTR with a PMC may be used as a method to promote and enhance measures of physical and emotional well being in cognitive rehabilitation for Traumatic Brain Injury (TBI). TBI is among the most frequent neurological disorders. TBI is a contributing factor to nearly a third (30.5%) of all injury-related deaths in the US.

Evidence-based practice guidelines from the Academy of Neurological Communication Disorders and Sciences recommend the use of external aids in the treatment of memory impairments as a rehabilitation practice guideline for adults with memory impairment following TBI. Practice guidelines from the European Federation of Neurological Societies recommend the use of “electronic external memory devices such as computers” as “probably effective aids for improving TBI or stroke patients' everyday activities.”

CTR with a PMC may be used as a method to promote and enhance measures of physical and emotional well being in cardiac rehabilitation to optimize physical function in people with cardiac disease or recent cardiac surgery. Cardiopulmonary or cardiac rehabilitation is a branch of rehabilitation medicine/physical therapy dealing with optimizing physical function in patients with cardiac disease or recent cardiac surgeries. Cardiac rehabilitation is often divided into phases that involve monitored exercise, nutritional counseling, emotional support, and support and education about lifestyle changes to reduce the risk of heart problems. The goals of cardiac rehabilitation are to help regain strength, to prevent worsening of existing heart conditions and reduce risk of future heart problems.

CTR with a PMC may be used as a method to promote and enhance measures of physical and emotional well being in physical rehabilitation, which is a medical specialty concerned with diagnosis, evaluation, and management of physical and/or cognitive impairment and disability. A PMC may contain personal recollections of institutionalized rehabilitation as well as the period preceding the related crisis, intended to prompt, or “prime,” forgotten memories, increase emotional awareness, cultivate social interaction and increase connectedness.

CTR with a PMC may be used as a method to strengthen military families for the physical, psychological, and emotional rigors of deployment. Deployments are tough on military families. Being apart for long periods, often in harm's way, makes the challenges of everyday family life even rougher. Difficulties arise for both service members and their families due to pre-deployment planning, family separation issues, stress, relocation, money management, spouse employment, parenting and child care, reunion and re-acclimation, and all too often, grief.

Military and other agencies are striving to find adequate measures to address these familial and organizational challenges. Counseling and other services have long been available to treat mental health problems once they develop.

Military families seem to handle stress and crises better when they handle them together. Since altering what they deal with during deployments is impractical, another approach is to change how and when they deal with deployment-related stressors. Performing CTR with a PMC may make that possible by: i) facilitating interpersonal connections with peers, family and friends; ii) reducing social isolation and anxiety associated with separation; iii) helping families adjust to deployment-related changes to family life; iv) promoting optimism problem-solving capacity and adaptive behaviors; v) improving self-esteem, and build self-efficacy; vi) strengthening hardiness, resistance and resiliency; vii) addressing the stigma of seeking treatment by preventing mental illness; and viii) boosting morale.

In addition to gathering memories, the system can be programmed to share relevant information families may find useful to their specific deployment phase, engagement, service or geographic location. Providing evidence-based, authoritative information early about what to expect can go a long way towards normalizing and coping; especially for families who have not yet endured a lengthy separation. Furthermore, it can help avert crises, minimize the need for command intervention or mental health counseling and possibly even reduce suicidal threats.

Using an unconventional approach like this has the potential to impact behavioral and emotional health without the stigma associated with conventional mental health treatment. The activity is enjoyable, helps families stay connected, and may also help manage the myriad issues that impact families while their warrior is away. At the conclusion of deployment, the PMC can be presented digitally for easy viewing and sharing, developed into a fully personalized website, or printed as a beautiful, full color book.

One embodiment of the present invention may comprise a tool for collaborative therapeutic reminiscence to enhance physical and emotional well-being that solicits a group of user's explicit and implicit memories of a specific occasion, event, time period, person or group of persons using nominal group recall; gathers and preserves digital media that represent group members' memories; analyzes and selects portions of preferred group members' content to compile a collaborative memory; presents selected components, or the entire compilation to the user to evoke potential changes to user's physical and emotional health; elicits development and recognition of latent memories; and facilitating memory reconsolidation by repeating the gather, preserve, and enhance process to permit contribution of previously latent memories that are now explicit memories; evokes potential changes to participants' physical and emotional health; and iteratively curates a tangible artifact of the reminiscence activity called a Personalized Memory Compilation (PMC), that may further promote therapeutic benefit and facilitate future recall.

An online application may enable a first user to navigate to a web site and enroll in the reminiscence exercise. Enrollment may also be done by a family member, work supervisor or health care practitioner, depending on the situation. To get started, name and contact data may be required (e.g., telephone number, email address).

Upon enrolling, the first user may be solicited to recall a specific occasion, event, time period, person or group of persons, and to provide details of such; the user may be presented with a series of variable questions or thought prompts, given a way to enter free form text, and invited to upload digital images, audio, or video.

The method may be practiced wherein a first user selects from a list of other users, or supplies the names and contact information for people the user desires to participate in reminiscence. Such people may be members of an existing group or ad-hoc group. Such users may be contacted by a printed and mailed invitation, an email, SMS text, telephone call, or other communication method to solicit contribution of their explicit and implicit memories using that medium or others.

The present invention may feature the gathering and preserving of digitally encoded audio, video, images, text or other media and/or other data (e.g., a letter from family or friends, a personal note from a colleague or service provider, comments and images sent by acquaintances, friends, or family, records of personal activity or performance) submitted by individuals who may be acquainted or related (e.g., members of the same company or military unit, undergoing rehabilitation at the same facility, being treated by the same clinician, and such). Such gathering may be performed using a technique called nominal group recall, wherein each group member contributes their content separately from the remainder of the group on a website. The website may provide a social networking style user interface (UI) that provides a platform that manages group member collaboration through online interaction via a suite of web based tools designed to facilitate better communication, better interaction and better quality and volume of information and data.

A memory compilation may be a collection of images, text, and other digital content that may be analyzed and brought together by a processor and a stylized template that may be customized by the application based on pre-arranged instructions. A memory compilation, or a portion of the memory compilation, may be made available at a given location (such as at a URL) that may be used, formally or informally, in a therapeutic fashion, shared with others, combined into a self-contained digital asset (such as, in an HTML5 or CSS layout), published to other web sites or web logs, printed as a hard- or soft-cover book, or maintained for future use by the user.

The present invention may contain a large portion of personalized and customized information unique to a user. There may be portions of the compilation that are the used by/for others' compilations (e.g., friends' profiles, locations, or organization information). One aspect of the present invention is that selected, relevant content may be used more than once and made available to other users based on personal preferences or pre-arranged criteria, so that a personalized memory compilation may be composed for more than one person based on information provided only once by each user.

Certain of the content may be provided by a different user and assembled in a page template by combining a plurality of content selected based upon pre-determined criteria, or by the other user, each characterized by a content-specific format and rendered in defined areas of the selected layout, within limits based upon the constraints of the content format. The other user may provide written responses to multiple, group-specific questions.

The method may also continue with determining at least one characteristic of at least one component of the content compilation and arranging responsive to the at least one characteristic, optionally arranging responsive to the content of the images or text. Other options for the method include providing the content in a first order and arranging the content into a second order different from the first order; changing the dimensions of content; grouping the selected content into a plurality of groups, and wherein arranging the content comprises arranging the content responsive to the grouping; and/or arranging irrespective of an order of acquisition of the content.

The present invention may comprise an interactive method in which digital content previously provided by the user may be used to select or generate questions or conversation prompts about a specific time period or life occasion based on pre-arranged criteria supplied by another user. The exercise may be designed to promote reminiscing about a specific person, relationship, event or era in the user's life and to identify and reinforce accomplishments and other positive aspects of the recalled experiences to evoke potential changes to user's physical and emotional health. The user may be prompted to supply answers to such questions, or journal about such prompts, either orally, in writing, or by using a supplied digital device. Answers, journal entries or other digital content supplied by the user may be evaluated by another user, preserved, and later reused in another similar interactive exercise, or combined with other digital content, provided by the user or other users, to create a unique, digital or printed, personalized memory compilation.

Analysis of a content compilation may be conducted to provide therapeutic feedback to the user or group of users. Such analysis may include a statistical analysis of activity such as: i) frequency and duration of participation; ii) expansion or contraction of locus of memories over time; iii) occurrence and frequency of repetition or contradiction; iv) increased/decreased communication. This data may provide family members or supervisors with insight into the user's condition that can be used to make modifications to the user's environment or activity.

A memory compilation may be presented in a variety of forms to evoke potential changes to user's physical and emotional health; elicit development and recognition of latent memories; and facilitate memory reconsolidation by re-initiating the gather, enhance, and preserve process to permit contribution of previously latent memories that are now explicit memories.

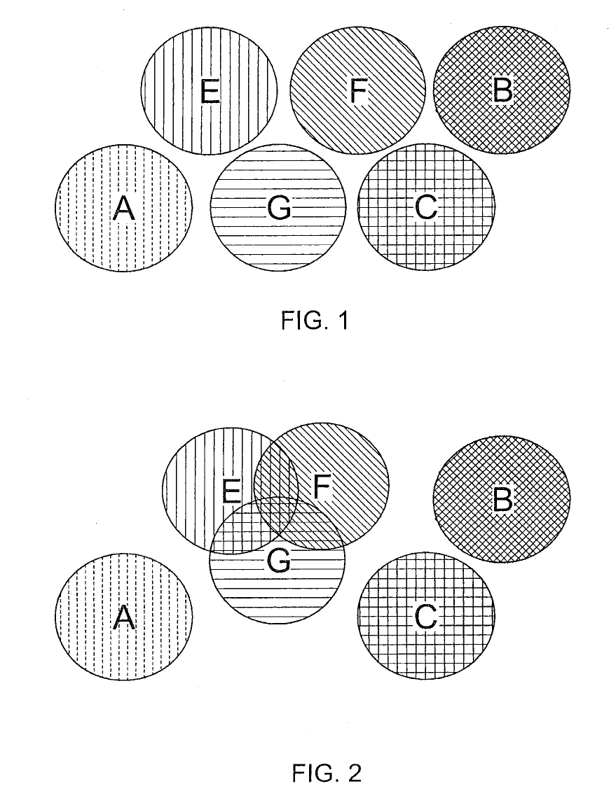

Referring now to the drawings in detail, wherein like reference characters refer to like elements, there is shown in FIG. 1 a plan diagram of group members' memories A, B, C, E, F, and G (in no particular sequence) sharing information separately. FIG. 1 may be considered to be a Venn diagram containing a set of elements representing the explicit memory provided by each group member. Items A, B, C, E, F, and G represent the memories of the respective group members. Each member's separate sharing of their respective memories with the group is termed nominal group recall.

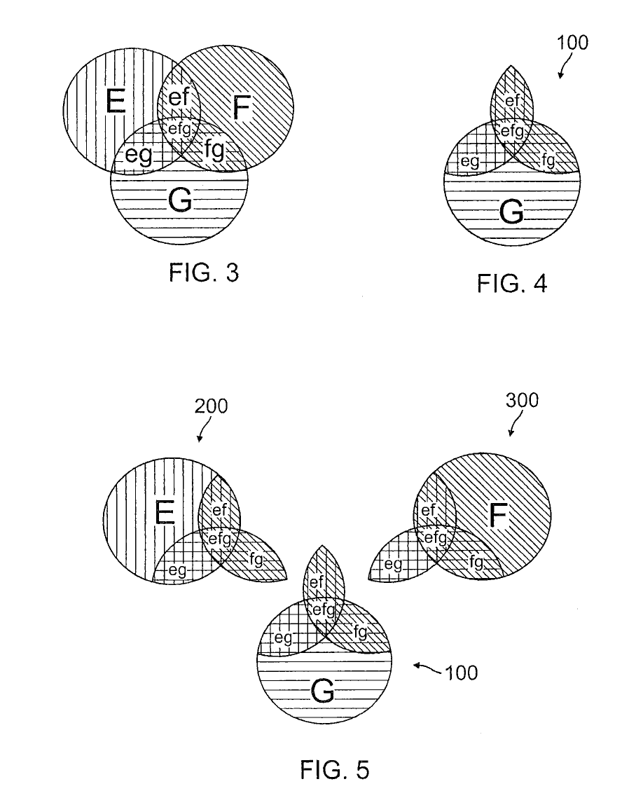

Selective inclusion is shown in FIG. 2, wherein, for example, memories from group members E, F, and G are selectively included while not necessarily including memories from, for example, group members A, B, and C. The selected group members E, F, and G are shown in FIG. 3 collaboratively sharing information, such as memories. Collaborative memory exists within the intersection of memories shared among the selected group members E, F, and G. The Venn diagram in FIG. 3 represents the collaborative memory of selectively included memories from group members E, F, and G according to an embodiment of the present invention. The union set of all memories provided by selectively included group members E, F, and G, contains shared memories, such as the intersection set of explicit memories from selectively included group members E, F, and G.

In the situation shown in FIG. 3, intersectional area of may represent intersecting or shared memories between members E and F. Likewise, intersectional area eg may represent intersecting or shared memories between members E and G. Regarding members F and G, intersectional area fg may represent intersecting or share memories between members F and G. Intersectional area efg may represent intersecting or shared memories among members E, F, and G.

The information or other type of content (such as memories) provided by each member may be enhanced by the collaborative output produced by the methods of the present invention. Group members may discover new information about a shared experience for which one or more selected members may have been unaware, remembered differently, experienced from another perspective, or otherwise have non-identical information.

Enhanced memory input may be seen in FIG. 4, which shows an isolated portion of FIG. 3, namely the information 100 available to member G, comprising intersectional information shared with member E (eg), intersectional information shared with member f (fg), intersectional information shared with members E and F (efg) plus intersectional information not originally possessed by G, but shared between members E and F (ef). The information quality available to member G is enhanced by the collaboration between members E and F. In one aspect, this could result in enhancement of member G's memory of an experience shared among members E, F, and G. Conceptually, the area of the circle representing member G's memory is enlarged by the additional portion ef added to the sum or memory originally shown for member G in FIG. 1 before the collaboration and enhancement of information.

Continuing with FIG. 4, each group member's memory now includes the collaborative memory (eg, fg, and efg) described in FIG. 3 as well as a latent memory (ef) elicited through the process of collaborative recall. The latent memory ef includes information shared by others (such as E and F) in the recall process about which the present member (G) was not previously aware.

As shown in FIG. 5 group members E and F may also retain enhanced memories, such as described above regarding member G in FIG. 4. The information 200 available to member E may be enhanced for producing output for the benefit of member E, while the information 300 available to member F may be enhanced for producing output for the benefit of member F. As demonstrated in the description and drawings referenced above, nominal group recall from FIG. 1 can proceed to selective inclusion in FIG. 2, collaboration in FIG. 3 to elicit latent memory, and producing an enhanced memory.

It should be understood that the above-mentioned group members A, B, C, although not described expressly as sharing memories with members E, F, and G, the above methods may occur among members A, B, C, or other members in parallel with the described interaction involving members E, F, and G, whether simultaneously, subsequently, prior to, or in any feasible sequence. Essentially, it is to be understood that the above examples are not limiting, especially as to the extent of interaction among group members or members of various sub-groups.

The collaborative output is a combination of all contributed explicit memory plus an emergent memory called latent memory.

Latent memory is not explicit memory, but is an emergent function of group recall. It comes into being only after participants share their explicit memories. As participants find new information about the shared experience and others provide information, about which individual participants may not recall or about which the individual participants were not previously aware. Represented graphically, latent memory lies at the intersection of two or more participant's explicit memory.

The significance of latent memory to the task of preserving memories comes from the capacity to alter how explicit memory is both perceived, and later, recalled. To preserve the most complete and accurate recollection of a shared event, the emergence of latent memory must be carefully managed. If explicit memories are combined too soon in the preservation process, then the development of latent memory may alter participants' recollection. Furthermore, because group recall has non-linear properties, the emergence of latent memory has the potential to permanently change more than one group member's recollection; an outcome potentially deleterious to an accuracy goal.

As group recall takes place, a collaborative memory Zₙ₊₁, is constructed iteratively as each participant contributes explicit shared memory and a latent shared memory emerges. Using this iterative formula, we can demonstrate the synergistic nature of group recall by comparing the collaborative output to the collective sum of the participants' contributions. Mathematically, as long as catalyst C is positive, a²+2a bi+b²+C will be greater than a². The corollary is, if catalyst C is sufficiently negative—in other words, there is sufficient noise or interference in the process—then a²+2a bi+b²+C could be less than a². In other words, if the process that catalyzes the iterative memory recall is not performed properly (a negative C value), the output could be deteriorated. Conversely, if that process is performed properly (a positive C value), it will produce synergy and symbiosis (i.e., a better memory).

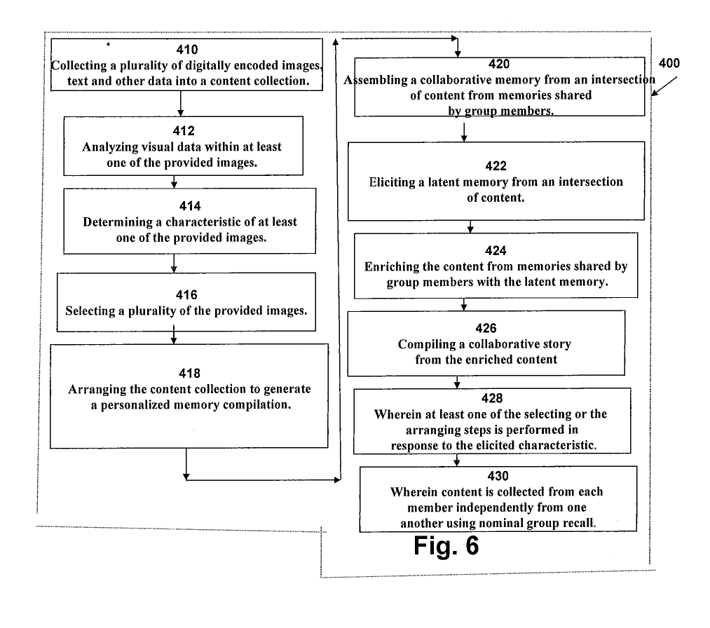

Referring to FIG. 6, a method 400 of the present invention may include a Step 410 of soliciting memories from members of a first group. Step 412 may comprise, collecting content from one or more data sources for the memories, such as digitally encoded images, text, audio, video and other data into a content collection. Another step, Step 414 may include identifying preferences of the members of the first group. A Step 416 may involve analyzing portions of the content collection to identify collaborative memories. Presenting the collaborative memories to a user for indication of social support, increasing social support, decreasing social support, and/or altering perceived social support may comprise Step 418. Step 420 may also comprise developing latent memories from collaboration between group members. A Step 422 may include reconsolidating memories from the content collection and the developed latent memories. Step 424 may involve compiling a collaborative output from the content collection. A portion of the content collection may be assembled into a collaborative memory of a second group.

It should be understood, of course, that the foregoing relates to exemplary embodiments of the invention and that modifications may be made without departing from the spirit and scope of the invention as set forth in the following claims. Furthermore, a method herein described may be performed in one or more sequences other than the sequence (e.g., order of steps) presented expressly in the claims or elsewhere herein.

We claim:

1. A method for eliciting memories, increasing emotional awareness, and cultivating social interaction to increase social connectedness comprising the acts of:
a) identifying a group of individuals who have memories of a common experience with an individual identified as a focus of therapy;
b) performing nominal group recall by, individually and independently, prompting the group members and the individual identified as the focus of the therapy to provide explicit memories of the common experience, wherein prompting the individual identified as the focus of the therapy comprises performing at least one act selected from the group consisting of:
i) presenting that individual with a series of variable questions or thought prompts;
ii) giving that individual a way to respond with free form text; and
iii) inviting that individual to upload digital images, audio or video;
c) eliciting latent memories, indicating social support, and evoking changes in the physical and/or emotional health of the individual identified as the focus of the therapy by sharing, with the group members and the individual identified as the focus of the therapy, a memory compilation comprising explicit memories of the common experience, wherein the memory compilation is created through a processor analyzing and bringing together memories of the common experience using a template customized based on the application of pre-arranged instructions;
d) reconsolidating the memories by repeating the acts of nominal group recall and latent memory elicitation at least one time;
e) creating a subsequent memory compilation for the individual identified as the focus of the therapy using memories which were provided, elicited and reconsolidated in the performance of acts (a)-(d).

2. The method of claim 1, wherein:
a) in at least one repetition of the performance of nominal group recall, using explicit memories previously provided by one user to select or generate prompts about the shared experience based on criteria provided by another user; and
b) in at least one repetition of the performance of eliciting latent memories, the memory compilation for that elicitation of latent memories comprises content provided by one user and assembled in one or more page templates using pre-determined criteria from another user, wherein each of the page templates is characterized by a content-specific format and rendered in a defined area of a selected layout within limits based upon constraints of the content-specific format.

3. The method of claim 1, comprising:
a) gathering data regarding interactions which take place during the prompting of explicit memories, elicitation of latent memories or indication of social support;
b) using the gathered data to generate recommendations regarding changes in the environment or activities of the individual identified as the focus of the therapy.
